(19) United States
(12) Patent Application Publication
(10) Pub. No.: US 2021/0079335 A1
(43) Pub. Date: Mar. 18, 2021

(54) APPARATUS AND METHOD FOR ENHANCING SPORULATION OF BACTERIA

(71) Applicant: Homebiotic, Inc., Silverdale, WA (US)

(72) Inventor: Steven John King, Victoria (CA)

(21) Appl. No.: 16/571,339

(22) Filed: Sep. 16, 2019

(51) Int. Cl. C12M 1/00; C12M 1/24; C12M 1/12

(52) U.S. Cl. CPC C12M 31/10; C12M 23/08; C12M 21/02; C12M 29/12; C12M 25/10; C12M 29/18; C12M 23/22

(57) ABSTRACT

An apparatus for enhancing bacterial sporulation comprising a laminar flow chamber including a plurality of laminar flow tubes that smooth flow of coolant, a coolant input port that is coupled to the laminar flow chamber that supplies the coolant to the laminar flow chamber from a coolant source, a transparent tube section that is coupled to the laminar flow chamber and includes light-emitting diode (“LED”) light modules, the transparent tube section receives the smoothed flow of coolant from the laminar flow chamber, a culture solution tube that traverses through the laminar flow chamber and the transparent tube section, wherein a portion of the culture solution tube that is within the transparent tube section is surrounded by the coolant and is exposed to light from the LED light modules, and a culture solution input port that is coupled to the culture solution tube and supplies a culture solution to the culture solution tube.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

### Field of the Invention

This application generally relates to devices that encourage bacterial sporulation, and in particular, devices that facilitate sporulation in bacteria by cooling and photomodulation.

### Description of the Related Art

A spore is a reproductive structure that is adapted for dispersal and survival for extended periods of time in unfavorable conditions. Spores form part of the life cycles of many organisms, such as bacteria, plants, algae and fungi. In a limited number of bacteria, spores can preserve the genetic material of the bacteria when conditions are inhospitable and lethal for the normal (vegetative) form of the bacteria. Relative to the norm life span of the microorganism, spores are designed to protect a bacterium from heat, dryness, and excess radiation for a long time.

Bacillus is a type of sporulating bacteria that is widely distributed in nature and that can be used in probiotics and biofilms. Scientifically, Bacillus species (“spp”) are gram-positive endospore forming bacteria. Bacillus spores are tolerant to extremes of environmental conditions including desiccation and heat. Spore germination leads to rapid growth of the vegetative (non-spore) form of the bacteria. Non-harmful (e.g., non-pathogenic, non-toxicogenic) species and strains of Bacillus are known and are “generally regarded as safe” (GRAS), and have found United States Environmental Protection Agency and United States Department of Agriculture approval for use in commercial processes. Bacillus bacteria are used in various fields such as production of enzymes and useful substances, production of fermented foods, decomposition of organic substances, microbial pesticides and microbial fertilizers.

However, Bacillus strains are difficult to commercialize without efficient sporulation ability. Thus, there has gone unmet a need for improved systems and methods for growing and producing Bacillus spores.

### SUMMARY OF THE INVENTION

The present invention provides a method and apparatus for enhancing bacterial sporulation. According to one embodiment, the apparatus comprises a laminar flow chamber including a plurality of laminar flow tubes that smooth flow of coolant, a coolant input port that is coupled to the laminar flow chamber that supplies the coolant to the laminar flow chamber from a coolant source, a transparent tube section that is coupled to the laminar flow chamber and includes light-emitting diode (“LED”) light modules, the transparent tube section receives the smoothed flow of coolant from the laminar flow chamber, a culture solution tube that traverses through the laminar flow chamber and the transparent tube section, wherein a portion of the culture solution tube that is within the transparent tube section is surrounded by the coolant and is exposed to light from the LED light modules, and a culture solution input port that is coupled to the culture solution tube and supplies a culture solution to the culture solution tube.

The apparatus may further comprise a coolant output port that is coupled to the transparent tube section and configured to allow the coolant to exit the apparatus. The apparatus may further comprise a culture solution output port that is coupled to the culture solution tube and configured to allow the culture solution to exit the apparatus. The apparatus may further comprise one or more sensors that are coupled to the culture solution output port to measure a rate of sporulation. The culture solution and the coolant may flow in a common axial direction. The transparent tube section may simultaneously cool the culture solution with the coolant and exposes the culture solution to light with specific wavelengths from the LED light modules at a given intensity that induces sporulation. In one embodiment, the coolant source includes a chiller unit. The culture solution tube may be constructed from borosilicate glass. The laminar flow chamber may comprise a chamber including laminar flow tubes that are sandwiched between layers of fibrous sponge-like material. The laminar flow tubes may comprise a plurality of small diameter tubes.

The LED light modules may comprise blue LEDs. The LED light modules may also comprise LEDs in an amount between 10 and 50 that are arranged in a given strip. The LED light modules may be arranged in one of vertical, horizontal, band or spiral wind arrangements. The LED light modules can be configured to pulse in increasing and/or varying intensity or brightness and duration. The LED light modules can be configured with a microcontroller to operate in one or more modes including constant, pulsed, and including one or more levels of intensity. The LED light modules may include LEDs that emit a wavelength between 400 nm and 425 nm. The LED light modules may also be configured according to programmable sequences including frequency and luminosity changes over time. In another embodiment, the LED light modules include ultraviolet (“UV”) LEDs that emit a wavelength between 275 nm and 300 nm. The culture solution may include Bacillus spp. The apparatus may further comprise a temperature sensor that is coupled to the coolant source and monitors a temperature of the culture solution to control the flow of coolant from the coolant source to a desired temperature.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 illustrates a sporulation enhancement apparatus according to an embodiment of the present invention.

FIG. 2 illustrates a cut-away view of the sporulation enhancement apparatus according to an embodiment of the present invention

FIG. 3 illustrates a sporulation enhancement apparatus according to another embodiment of the present invention.

FIG. 4 illustrates a sporulation enhancement apparatus including a LED light module spiral according to an embodiment of the present invention.

FIGS. 5 through 7 illustrate a sporulation enhancement apparatus in a flat plate construction according to an embodiment of the present invention.

FIG. 8 presents a sporulation enhancement apparatus in a cylindrical construction according to an embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. The following detailed description is, therefore, not intended to be taken in a limiting sense.

The present application discloses an apparatus and method for inducing increased rates of sporulation of bacteria (e.g., Bacillus spp) after it is filtered out of a growing medium. The apparatus may include a combination of an axial flow chiller and light-based treatment. According to one embodiment, the apparatus may comprise a variable/programmable apparatus that uses blue light exposure (an environmental stressor) to increase the rate of sporulation in a bacterial culture solution that is circulated through the apparatus. The bacteria may be photomodulated by the blue light exposure which activates sporulation activity. The apparatus may expose the filtered culture solution to a combination of low temperature (e.g., 4° C. or approximately 39.2° F.) via chilled coolant and low intensity blue light from an array of light-emitting diode (“LED”) modules. The removal of nutrients (by filtering the bacteria from the growing medium) and lowered temperature induce sporulation, which may be enhanced by the addition of a blue light in the 400-425 nm range, for example.

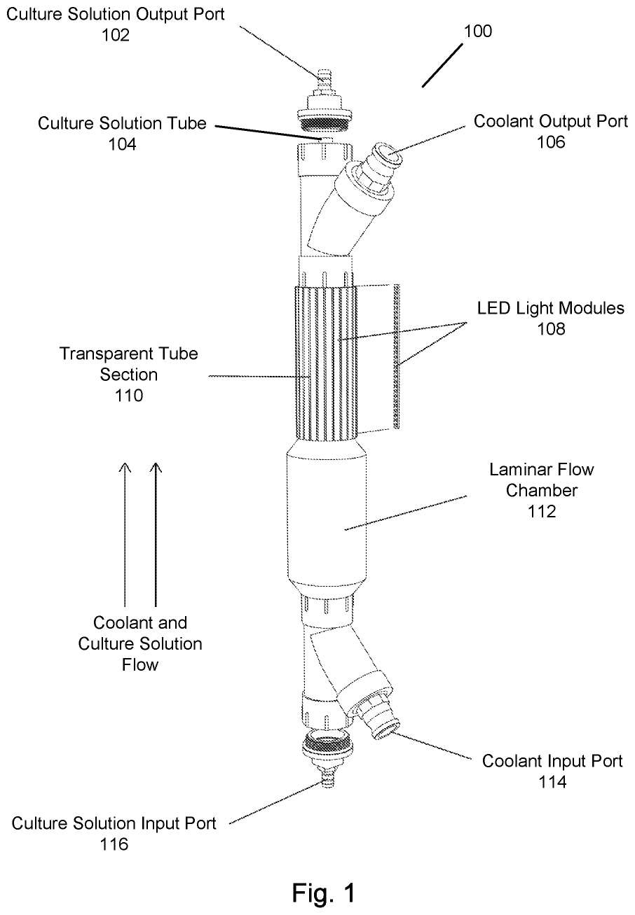

FIG. 1 presents a sporulation enhancement apparatus according to an embodiment of the present invention. The apparatus 100 may include a co-axial flow configuration using laminar flow of coolant (e.g., water) to achieve chilling and light-based treatment of a culture solution simultaneously. The culture solution may include bacteria filtered from a growing medium (e.g., Bacillus spp). The apparatus 100 simultaneously chills the culture solution while exposing it to light with specific wavelengths at a low intensity to induce more rapid sporulation by creating an increasingly hostile environment for active bacteria.

The apparatus 100 may comprise an input side where the culture solution enters the apparatus 100 through culture solution input port 116 and the coolant enters the apparatus 100 at coolant input port 114 such that the culture solution and coolant flow in a common axial direction. The coolant may be supplied from a chiller unit (not illustrated) that is coupled to the coolant input port 114. The culture solution input port 116 may be coupled to a culture solution tube 104 to supply the culture solution to the culture solution tube 104. The culture solution input tube 104 may be configured in the center of the apparatus 100. The culture solution tube 104 may be constructed from glass (or any other non-reactive transparent material) and runs along a length (centered) of the apparatus 100 (e.g., from culture solution input port 116 to culture solution output port 102). For example, thin borosilicate glass may be used for the culture solution tube 104 may increase heat transfer and result in faster chilling and longer duration at a target temperature. The culture solution may be transported by the culture solution tube 104 to pass through a laminar flow chamber 112 and a transparent tube section 110.

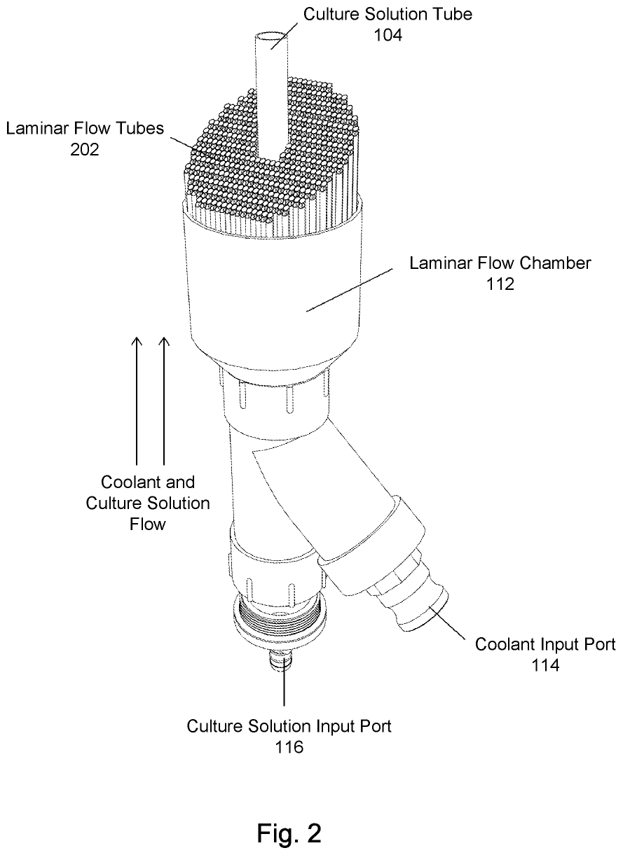

Coolant from the coolant input port 114 flows through the laminar flow chamber 112 to smooth the flow. FIG. 2 presents a cut-away view of the sporulation enhancement apparatus according to an embodiment of the present invention. The laminar flow chamber 112 may comprise a chamber filled with laminar flow tubes 202 that are sandwiched between layers of fibrous sponge-like material (not illustrated). The laminar flow tubes 202 may comprise a plurality of small diameter tubes that may be approximately the size and shape ranging from small drinking straws to coffee stir sticks. The laminar flow chamber 112 may smooth the coolant flow sufficiently such that there are virtually no distortions resulting from coolant flow that would diminish the effects of light treatment. That is, the laminar flow chamber 112 may reduce bubbles and turbulence in the coolant which could otherwise reduce light transmission when passing light through the coolant to the culture solution tube 104.

Referring again to FIG. 1, after passing through the laminar flow chamber 112, the coolant may be transported to a transparent tube section 110. The transparent tube section 110 may be filled with the coolant, inside of which is the culture solution tube 104 which the culture solution flows through. The culture solution tube 104 may be surrounded by the coolant in the transparent tube section 110 to chill the culture solution in the culture solution tube 104 to a desired temperature (e.g., 4° C. or approximately 39.2° F.). Temperature of the culture solution may be monitored by a temperature sensor that may be embedded within or coupled to culture solution tube 104. According to one embodiment, the temperature sensor may be connected to or communicate with the coolant source to control the temperature or flow of the coolant to a given temperature.

Transparent tube section 110 may be enveloped by LED light modules 108 to expose the culture solution of the culture solution tube 104 to specific amounts of light. After passing through transparent tube section 110, the coolant may exit the apparatus 100 from coolant output port 106 and return to a chiller for recirculation back to coolant input port 114. The culture solution may exit the apparatus 100 from the culture solution tube 104 through culture solution output port 102 for sampling and/or recirculation back to the culture solution input port 116. Progress may be measured via sensors attached to the solution output port 102 or via grab samples for microbiological analysis to determine the rate of sporulation, which may indicate when the the culture solution has achieved maximum sporulation.

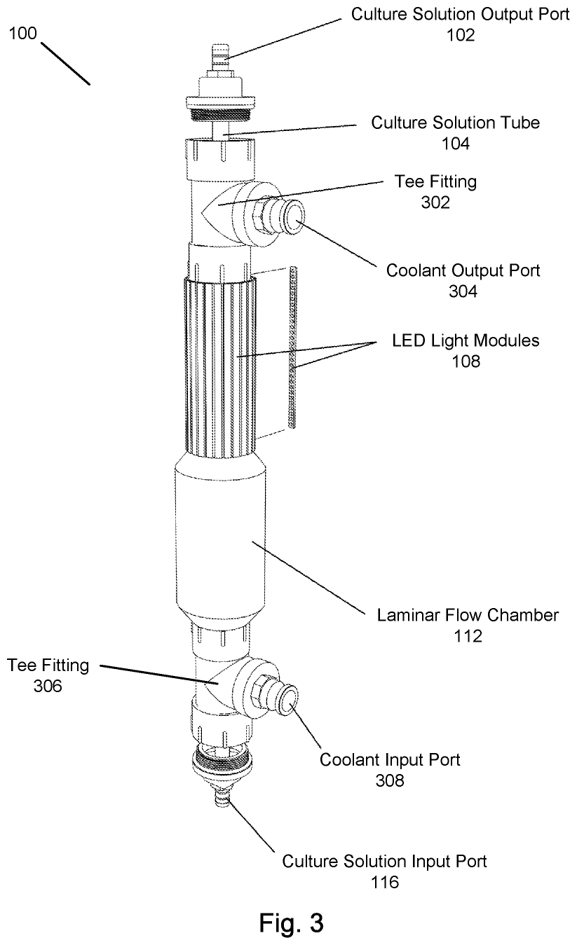

Coolant output port 106 and coolant input port 114 may be connected to the apparatus 100 with wye fittings to allow for smoother coolant flow and quieter operation as the coolant avoids making sharp turns upon entering or exiting the apparatus 100. The angle of the coolant input port and the coolant output port may be varied, for example, to allow for better connection access. FIG. 3 presents an alternative embodiment of the sporulation enhancement apparatus where coolant output port 304 may be connected to the apparatus 100 via tee fitting 302 and coolant input port 308 may be connected to the apparatus 100 via tee fitting 306.

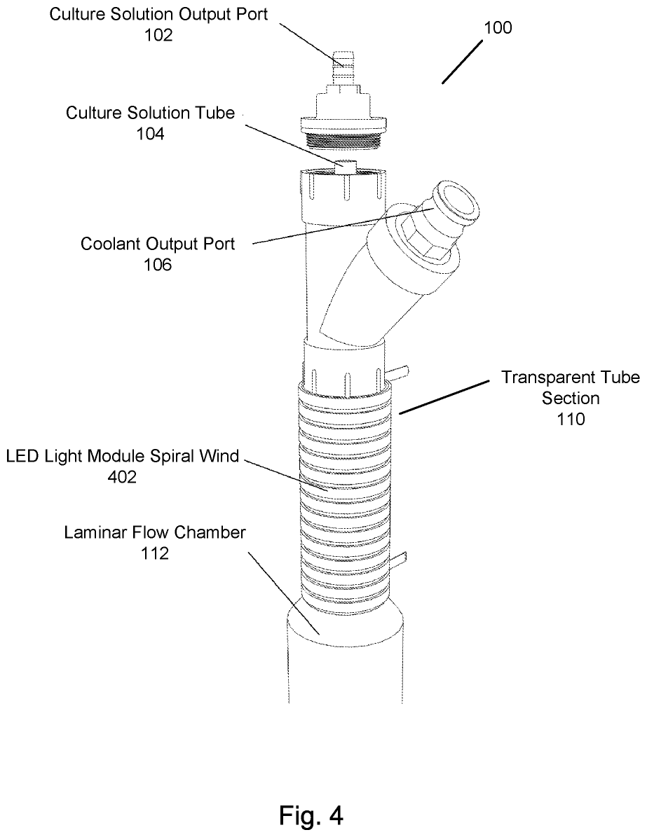

The LED light modules 108 may comprise strips of blue LEDs. The LEDs of the LED light modules 108 may be arranged in strips having from about 10 to about 50 LEDs per strip. Strips of various sizes and dimensions may be used. Vertical and horizontal band or spiral wind arrangements can be used as options for LED placement. For example, FIG. 4 illustrates a sporulation enhancement apparatus including a LED light module spiral wind 402 that surrounds transparent tube section 110

LED light modules 108 may be configured to pulse in increasing and/or varying intensity/brightness and duration. The pulsed light passes to the culture solution tube through the coolant, which may have the benefit of carrying away any heat that the LEDs may generate. Light wavelength emitted from LED light modules 108 may be between 400-425 nm. The LED light modules 108 can be operated (either individual elements or collectively) in a variety of modes, including constant and pulsed, both with varying levels of light intensity, driven by a microcontroller. The pulse intensity and duration of light exposure can be varied for each bacterial species and strain to obtain the optimum operating conditions. Presets can be programmed into the microcontroller. Programmed sequences, including frequency and luminosity changes over time can also be used, for more delicate operation.

According to an alternative embodiment, ultraviolet (“UV”) LEDs in the 275-300 nm range may be interspersed with the blue LEDs in LED light modules 108 to increase microbial stress. This range of ultraviolet light is off the peak germicidal frequency of 265 nm, as the intent is to stress rather than to kill the bacteria. The ultraviolet LEDs may operate at a lower intensity than the blue LEDs and may also be individually controlled via the microcontroller. For energy efficiency, the sporulation enhancement apparatus may also be covered with an insulating wrap. For example, insulating the exterior housing of the apparatus may improve energy efficiency by reducing the amount of ambient heat absorbed from the environmental surroundings. This may also shield the operator from light leaking out of the apparatus, which may be a visual irritant and/or minor ionizing radiation hazard when used in conjunction with UV-C light modules.

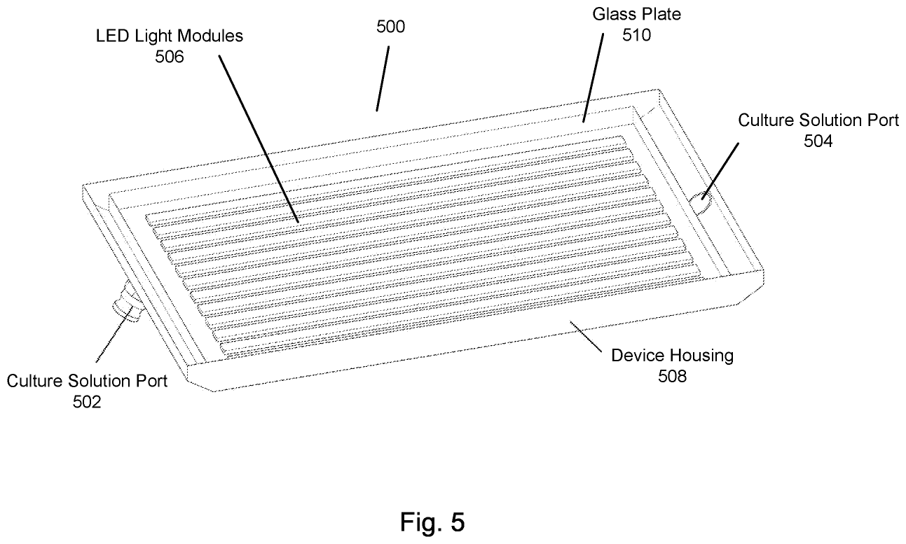

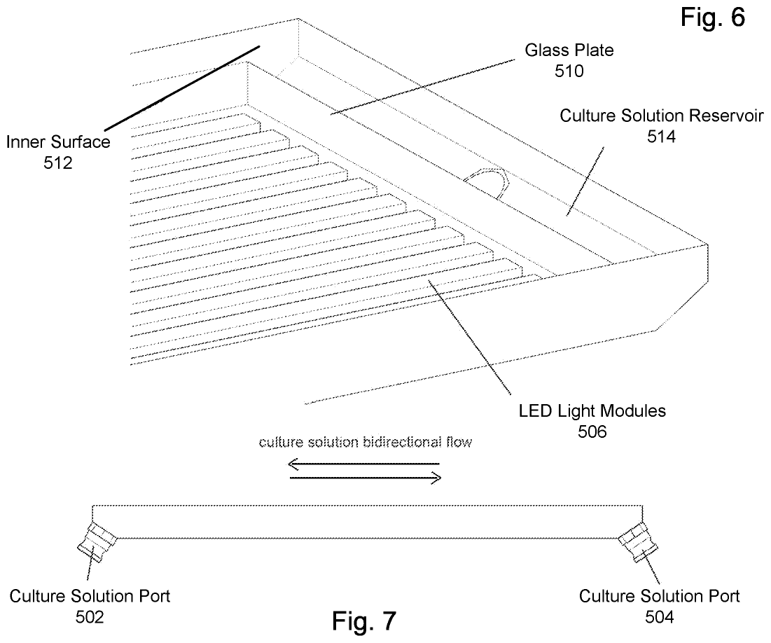

FIGS. 5 through 7 present a sporulation enhancement apparatus according to an alternative embodiment of the present invention. Apparatus 500 comprises a light-based flat plate configuration for treating a culture solution that may be pre-chilled before entering the apparatus. LED light modules 506 may be mounted on under a glass layer on a bottom of glass plate 510. The pre-chilled culture solution may be pumped from one of or either culture solution port 502 or culture solution port 504 into a culture solution reservoir 514. The apparatus 500 may support bidirectional flow of the culture solution such that the culture solution may be circulated to flow in either direction.

The culture solution reservoir 514 may comprise a channel formed along the sides between the device housing 508 and glass plate 510. As the culture solution reservoir 514 is filled with the culture solution, the culture solution will flow upward and over the glass plate 510. Culture solution flowing over the glass plate 510 is exposed to light emitted from the LED light modules 506 beneath the glass plate 510. The inner surface 512 of the apparatus housing can be mirrored or white, for maximum light efficiency. The culture solution may exit from one of or either culture solution port 502 and culture solution port 504 and recirculated to ensure maximum sporulation is achieved.

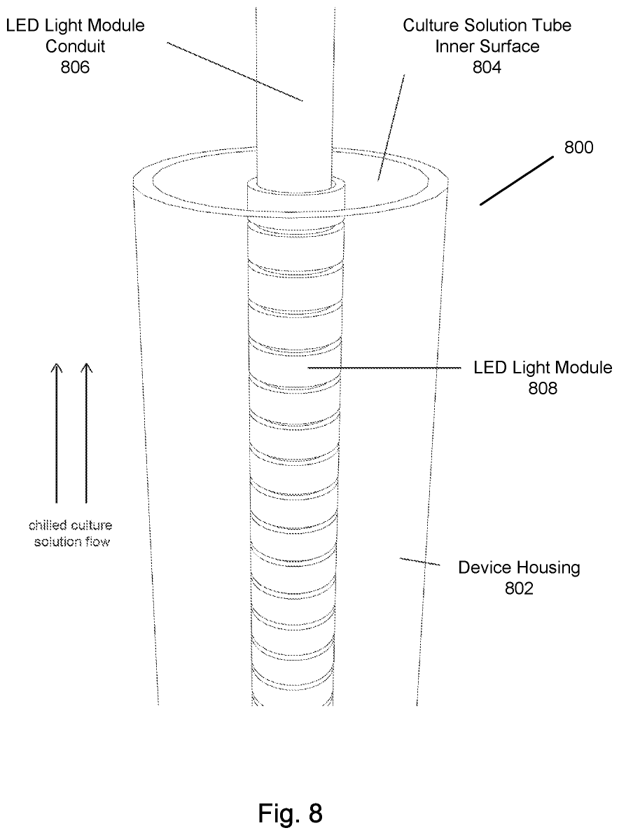

FIG. 8 presents a sporulation enhancement apparatus in a cylindrical construction according to another embodiment of the present invention. Apparatus 800 may comprise a device housing 802 including a culture solution tube inner surface 804 for culture solution to traverse through. A LED light module 808 may be attached to and supported by LED light module conduit 806 in the inner center of the device housing 802 where the culture solution may flow around them. The LED light module conduit 806 may comprise a tube including wiring for powering the LED light module 808. LED light module 808 may be constructed in vertical, horizontal and spiral wind light configurations, with a variety of distances between the LED light module 808 and the culture solution tube inner surface 804. In one embodiment, The LED light module 808 may also be encased in a transparent cylinder to isolate it from the culture solution.

FIGS. 1 through 8 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

1. An apparatus for enhancing bacterial sporulation,
the apparatus comprising:
a laminar flow chamber including a plurality of laminar flow tubes that smooth flow of coolant;
a coolant input port that is coupled to the laminar flow chamber that supplies the coolant to the laminar flow chamber from a coolant source;
a transparent tube section that is coupled to the laminar flow chamber and includes light-emitting diode (“LED”) light modules, the transparent tube section being configured to receive the smoothed flow of coolant from the laminar flow chamber;
a culture solution tube that traverses through the laminar flow chamber and the transparent tube section, wherein a portion of the culture solution tube that is within the transparent tube section is configured to be surrounded by the coolant and exposed to light from the LED light modules; and
a culture solution input port that is coupled to the culture solution tube and is configured to supply a culture solution to the culture solution tube.

2. The apparatus of claim 1, further comprising a coolant output port that is coupled to the transparent tube section and configured to allow the coolant to exit the apparatus.

3. The apparatus of claim 1, further comprising a culture solution output port that is coupled to the culture solution tube and configured to allow the culture solution to exit the apparatus.

4. The apparatus of claim 3, further comprising one or more sensors that are coupled to the culture solution output port to measure a rate of sporulation.

5. The apparatus of claim 1, wherein the culture solution and the coolant flow in a common axial direction.

6. The apparatus of claim 1, wherein the transparent tube section simultaneously cools the culture solution with the coolant and exposes the culture solution to light from the LED light modules at a given intensity that induces sporulation.

7. The apparatus of claim 1, wherein the coolant source includes a chiller unit.

8. The apparatus of claim 1, wherein the culture solution tube is constructed from borosilicate glass.

9. The apparatus of claim 1, wherein the laminar flow chamber comprises a chamber including laminar flow tubes that are sandwiched between layers of fibrous sponge-like material.

10. The apparatus of claim 9, wherein the laminar flow tubes comprise a plurality of small diameter tubes.

11. The apparatus of claim 1, wherein the LED light modules comprise blue LEDs.

12. The apparatus of claim 1, wherein the LED light modules comprise LEDs in an amount between 10 and 50 that are arranged in a given strip.

13. The apparatus of claim 1, wherein the LED light modules are arranged in one of vertical, horizontal, band or spiral wind arrangements.

14. The apparatus of claim 1, wherein the LED light modules are configured to pulse in increasing or varying intensity or brightness and duration.

15. The apparatus of claim 1, wherein the LED light modules are configured with a microcontroller to operate in one or more modes including constant, pulsed, and including one or more levels of intensity.

16. The apparatus of claim 1, wherein the LED light modules include LEDs that emit a wavelength between 400 nm and 425 nm.

17. The apparatus of claim 1, wherein the LED light modules are configured according to programmable sequences including frequency and luminosity changes over time.

18. The apparatus of claim 1, wherein the LED light modules include ultraviolet (“UV”) LEDs that emit a wavelength between 275 nm and 300 nm.

19. The apparatus of claim 1, wherein the culture solution includes Bacillus spp.

20. The apparatus of claim 1, further comprising a temperature sensor that is coupled to the coolant source and configured to monitor a temperature of the culture solution to control the flow of coolant from the coolant source to a desired temperature.
